(19) United States
(12) Patent Application Publication
(10) Pub. No.: US 2009/0253712 A1
(43) Pub. Date: Oct. 8, 2009

(54) AQUEOUS SOLVENT SYSTEM FOR SOLUBILIZATION OF AZOLE COMPOUNDS

(75) Inventors: Kristof KOVACS, Budapest (HU); Krisztina LUDANYI, Telki (HU); Gyorgy STAMPF, Budapest (HU); Istvan ANTAL, Budapest (HU); Imre KLEBOVICH, Budapest (HU)

Correspondence Address: YOUNG & THOMPSON, 209 Madison Street, Suite 500, ALEXANDRIA, VA, 22314, US

(73) Assignee: SEMMELWEIS EGYETEM, Budapest (HU)

(21) Appl. No.: 12/417,874

(22) Filed: Apr. 3, 2009

Related U.S. Application Data
(60) Provisional application No. 61/041,930, filed on Apr. 3, 2008.

(51) Int. Cl. A61K 31/496; A61K 31/4164; A61K 47/10; A61K 47/14

(52) U.S. Cl. 514/254.07; 514/399; 514/772; 514/785

(57) ABSTRACT

A pharmaceutical composition includes:

an azole derivative solubilized in a solvent system including: a) water, b) pH-adjuster, c) co-solvent and d) surfactant.

### FIELD OF THE INVENTION

This invention relates to an aqueous solvent system exerting synergistic solubilizing effect on hydrophobic azole derivatives and the preparation of compositions based on said solvent system. The composition containing an azole antifungal compound can be used in the treatment of systemic fungal infections, especially in parenteral (e.g. i.v.) dosage form.

### BACKGROUND OF THE INVENTION

Inadequate aqueous solubility of therapeutically active agents is a great issue in pharmaceutical development. Unfortunately a wide range of chemicals exhibit very poor aqueous solubility. Since a liquid dosage form should contain the therapeutically effective amount of the ingredient, the formulation of such compositions is a great practical problem in most cases. Consequently, the need to administer poorly soluble drugs in liquid dosage forms has been met by many scientists and several methods of formulating parenteral or oral medications of poorly soluble chemicals are known in the art. Some of these techniques are for example formulations of liposomes, emulsions, microemulsions, nanoemulsions or complex formation with cyclodextrins, hydrotropic solubilization, and formation of prodrugs. Other than these rather complicated approaches to increasing solubility, more conventional ways of increasing solubility are for example adjusting the pH of the solution, using organic co-solvents or micellization. A large proportion of approved parenteral and oral dosage forms utilize co-solvents and surfactants such as ethanol, propylene glycol, glycerol, polyethylene glycol, DMSO and polyethylene glycol sorbitan esters (polysorbates) for solubilization. Monistat® (or Daktarin®) was a concentrate for infusion containing miconazole as the active agent and Cremophor EL® surfactant as the solubilizing excipient. Complex formation with cyclodextrins, more particularly hydroxypropyl-β-cyclodextrin, as in the case of Sporanox® (Janssen Pharmaceutica Products, L.P.) lead to a solution containing therapeutically effective amounts of itraconazole for both oral and intravenous administration. Both miconazole and itraconazole are potent antifungal agents belonging to the group of azoles.

Fungi can cause either superficial or systemic infections. Superficial fungal infections are very common and their treatment is performed utilizing dosage forms intended for such diseases. Systemic fungal infections were less frequent, but over the past years an increasing incidence of systemic mycoses was observed world-wide. This correlates with the ever growing number of immune-compromised patients and the chronic use of antimicrobial agents. The HIV infected, patients receiving anticancer therapy or immune suppressive drugs and the elderly are most endangered. For example, it is estimated that the cause of death of 10 to 30% of AIDS patients is due to infections inflicted by Cryptococcus. The infections caused by these so called opportunistic fungal organisms vary greatly, but are usually more severe than superficial infections. Generally these infections are chronic and develop slowly as in the case of coccidioidomycosis, where primarily an acute respiratory disease develops followed by a chronic, often fatal infection of the skin, lymph glands, spleen and the liver. Other infections offer different courses and may cause infections of the kidney, liver, spleen, heart, eyes, brain, prostate, bone and other infections. In other cases, as in hospitalized patients mycoses may occur as iatrogenic infections, for example due to the insertion of catheters. In any case, these infections cause severe diseases and are to be dealt with.

Although these data are well known, combating systemic fungal infections continues to pose problems to physicians. It is also known, that compared to the wide range of antimicrobial agents, the amount of medications applicable in systemic mycoses is quite limited. Only ten active agents are approved in the US. The lack of medications and the fact that new resistant fungi are emerging demands the formulation of new compositions.

Azoles (U.S. Pat. No. 3,717,655) are a group of active pharmaceutical ingredients mainly built up of a five-membered nitrogen heterocyclic ring containing at least one other non-carbon atom, selected from the group of nitrogen, sulfur and oxygen, and contain side chains. They have been shown to be effective fungicidal and fungistatic agents against various fungi. Both of their effects, the killing of fungus and the stopping of the growth of fungus can be attributed to their ability to inhibit sterol 14-α-demethylase, a microsomal cytochrome P₄₅₀-dependent enzyme system. This results in reduced ergosterol synthesis and an excess of 14-α-demethylase sterols, which in turn disrupts the close packing of acyl chains of phospholipids and impairs the function of some membrane-bound enzyme systems. Fungi which are susceptible to azoles are fungi that are most commonly recovered from patients suffering from such microbial infections. These include Blastomyces, Histoplasma, Coccidioides, Aspergillus and Candida, other significant fungi include Cryptococcus, Torulopsis, Paracoccidioides, Rhizopus and Mucorand.

As stated above, azoles are a group of antifungal agents that can be used in the treatment of systemic fungal infections. Although these compounds are potent molecules, most of them show poor water solubility, thus formulation of a composition, which comprises therapeutically effective quantities of active agent, is difficult. To overcome this problem several patents and articles have been published over the years.

For example U.S. Pat. No. 4,861,580 discloses various compositions comprising of a poorly water-soluble therapeutically active agent, for example miconazole with different proportions of cholesterol hemisuccinate (tris salt) and D-alpha-tocopherol hemisuccinate (Tris salt). This liposome composition is capable of solubilizing various amounts of active agents. As with all liposomal products its disadvantage is to be found in its stability, both long term and in production, and in the complexity of its production and its very high cost.

As already stated before, the use of cyclodextrins as carriers for poorly soluble molecules can be applied to solubilize, for example azoles. Other than the previously mentioned Sporanox® (Janssen Pharmaceutica Products, L.P., U.S. Pat. No. 4,727,064) several other patents deal with the complexing of active agents with cyclodextrins. US Patent Application No. 2007/0082870 discloses the solubilization of itraconazole, voriconazole, ketoconazole and clotrimazole with the help of complex formation with hydroxybutenyl cyclodextrins. Miconazole was also solubilized by cyclodextrins, disclosed in the International Journal of Pharmaceutics 169 (1998) 15-22, under the title “Development of a non-surfactant parenteral formulation of miconazole by the use of cyclodextrins”. In all of the above examples cyclodextrins or derivatives thereof proved to be powerful solubility enhancers of the studied therapeutic agents. On the other hand, as with liposomes, cyclodextrins are very costly and their production is complex, especially when in need of cyclodextrins, which are to be administered parenterally.

Other means of solubilizing are for example the formation of emulsions (Patent number WO91/07962 and U.S. Pat. No. 5,651,991), microemulsions (U.S. Pat. No. 5,478,860), nanoemulsions (US Patent Application No. 2006/0292186), submicron suspensions (US Patent Application No. 2005/0048126) are also applicable means of solubilizing azoles, but the examples detailed in these inventions are rarely concerned about azoles. The main disadvantage of these pharmaceutical dosage forms is their stability during sterilization and also the complexity of their production.

To overcome these difficulties encountered in the previously listed methods of solubilizing, some inventions and articles deal with the possibility of solubilizing applying acids, co-solvents and surfactants separately or in combination. U.S. Pat. No. 6,100,285, a work that uses only acids as solubilizer, teaches the solubilization of itraconazole using various concentrations of glacial acid.

The use of co-solvents for the solubilization of poorly soluble compounds is disclosed in U.S. Pat. No. 6,361,758. This patent teaches a composition comprising of two co-solvents, an alcohol and a glycol for the solubilization of preferably 19-nor-1α,3β,25-trihydroxy-9,10-secoergosta-5,7(E),22(E)-triene. Said composition can be sterilized and may be administered parenterally.

Binary compositions for example acids and co-solvents are also used for improving the solubility of therapeutically active agents. U.S. Pat. No. 5,858,999 discloses a sterile aqueous pharmaceutical composition for parenteral administration comprising of a lazaroid, an acid for the adjustment of pH and a co-solvent.

An example of surfactants used alone or in combination with co-solvents is disclosed in US Patent Application No. 2004/0167152. Said patent application teaches and claims a solubility enhancing composition of CCI-779 comprising of stated active agent, an alcoholic solvent, an antioxidant, a diluent solvent and a surfactant. This composition, aside of being capable of dissolving therapeutically effective amounts of active agent, is a parenterally acceptable solvent system.

U.S. Pat. No. 5,461,068 teaches a composition containing an azole antifungal agent or its salt in a dermatological solution. Although the solvent system comprises a carboxylic acid, a polar solvent, a solubilizing agent and a surfactant, this composition is not for parenteral administration.

U.S. Pat. No. 4,912,124 again comprises a polar solvent, a solubilizing agent and a surfactant along with an azole derivative, but as in the previous patent, this solution is also a topical one not a solvent system for parenteral administration.

U.S. Pat. No. 6,383,471 discloses several compositions comprising a hydrophobic therapeutically active agent containing at least one ionizable functional group, an ionizing agent, a solubilizer and a surfactant and the method for producing the same. A few examples using itraconazole and tretinoin as the hydrophobic therapeutically active agent are disclosed and their stability was studied upon dilution with gastric and intestinal fluid. The exemplified solutions are as follows:

### EXAMPLE NO. 27

| | |
|---|---|
| Concentrated Phosphoric Acid | 0.025 g |
| Tween 20 | 0.300 g |
| Arlacel 186 | 0.200 g |
| Sodium Taurocholeate | 0.150 g |
| Propylene Glycol | 0.300 g |

### EXAMPLE NO. 28

| | |
|---|---|
| Concentrated Hydrochloric Acid | 0.025 g |
| Tween 20 | 0.300 g |
| Arlacel 186 | 0.175 g |
| Sodium Taurocholeate | 0.150 g |
| Propylene Glycol | 0.300 g |

### EXAMPLE NO. 29

| | |
|---|---|
| Concentrated Hydrochloric Acid | 0.025 g |
| Tween 20 | 0.300 g |
| Arlacel 186 | 0.150 g |
| Sodium Taurocholeate | 0.150 g |
| Propylene Glycol | 0.325 g |

### EXAMPLE NO. 30

| | |
|---|---|
| Concentrated Phosphoric Acid | 0.100 g |
| Tween 20 | 0.300 g |
| Sodium Taurocholeate | 0.100 g |
| Glycofurol | 0.500 g |
| Ethanol | 0.100 g |

### EXAMPLE NO. 31

| | |
|---|---|
| Concentrated Phosphoric Acid | 0.100 g |
| Tween 20 | 0.300 g |
| Arlacel 186 | 0.050 g |
| Sodium Taurocholeate | 0.100 g |
| Glycofurol | 0.500 g |
| Ethanol | 0.100 g |

Another method of solubilizing miconazole with the help of surfactant was applied in the previously mentioned Monistat® or Daktarin® intravenous solution. Polyoxyethylene-hardened castor oil (Cremophor EL®) was used to achieve a solution containing 10 mg/ml of miconazole. As written in U.S. Pat. No. 5,651,991, it was recently found that this surface active agent HCO-60 (Cremophor EL®) caused anaphylaxic shock and was questionable as an additive to drugs. Therefore, its preparations involve problems in that the clinical use is strictly restricted and hence, medical treatment cannot be sufficient.

The previously raised issues and the fact that therapeutically effective parenteral and oral formulations are needed for the treatment of systemic mycoses led us to the elaboration of the present invention. The solvent system applied in the present invention shows a synergetic effect compared to some very similar mixtures and to the known mixtures applied in the above mentioned patents, so the invention cannot be considered evident to a skilled person on the basis of the prior art.

### SUMMARY OF THE INVENTION

The objective of the present invention is to provide a pharmaceutically acceptable aqueous solvent system capable of dissolving therapeutically active agents, more preferably sparingly, slightly, very slightly soluble or practically insoluble or non-soluble therapeutically active agents. According to the present invention the said solvent system comprises water, a pH-adjuster (an acid or a base), a co-solvent and a surfactant.

Another objective of the invention is to formulate a composition for oral or parenteral administration, comprising a therapeutically active agent in the above mentioned solvent system.

It is another objective of the invention is a method for treatment of animals, more preferably mammals, even more preferably humans, comprising the administration of the above composition to the subject to be treated.

In an embodiment of the present invention the therapeutically active agent is selected from the group of azole antifungals. In an another embodiment of the present invention the azole antifungal agent can be e.g. azanidazole, bifonazole, butoconazole, clomidazole, clotrimazole, eberconazole, econazole, fluconazole, flutrimazole, genaconazole itraconazole, ketoconazole, miconazole, omoconazole, omidazole, oxiconazole, posaconazole, ravuconazole, saperconazole, sertaconazole, sulconazole, terconazole, tiabendazole, tioconazole, voriconazole or its salt. In a preferred embodiment the antifungal agent is ketoconazole or miconazole, or their salts.

In the present invention the solvent system exhibits a synergistic effect on the solubility of the therapeutically active agent. The synergistic effect means that the solubilizing effect of the solvent system (pH-adjuster, co-solvent and surfactant) is more than the sum of the solubilizing effect of each component separately.

Other advantages of the present invention will become apparent from the following detailed description of the invention, but it is to be understood that both the foregoing description and the following description serve to explain the understanding and realization of the invention and are not by any means restrictive to the scope of the invention.

### DETAILED DESCRIPTION OF THE INVENTION

Our experiments led us to a non-excepted finding of an aqueous solvent system capable of dissolving hydrophobic pharmaceutically active agents in therapeutically effective amounts. The substantial solubilizing effect of these solvent systems is based on the synergistic solubilizing effect of the applied ingredients. The compositions which are based on the present solvent system comprise a therapeutically active agent and the solvent system. The solvent system comprises water, a pH-adjuster (i.e. an acid or a base), a co-solvent and a surfactant. These solvent system are readily capable of solubilizing various types of active agents which are hydrophobic, i.e. sparingly, slightly, very slightly soluble or practically insoluble, or non-soluble in water.

The pharmaceutically active agent can be any azole type molecule (azole derivative). Preferably the active agent is an antifungal agent. Such azole derivatives are for example azanidazole, bifonazole, butoconazole, clomidazole, clotrimazole, eberconazole, econazole, fluconazole, flutrimazole, genaconazole itraconazole, ketoconazole, miconazole, omoconazole, ornidazole, oxiconazole, posaconazole, ravuconazole, saperconazole, sertaconazole, sulconazole, terconazole, tiabendazole, tioconazole, voriconazole or their salts. Even more preferably the antifungal agent is ketoconazole or miconazole or their salts. It should be understood that the list is merely illustrative and any analogue or derivative or a mixture of the stated molecules are included within the scope of the present invention.

The solvent system that is able to solubilize and keep in solution the previously mentioned therapeutically active agents is a complex solution comprising of water, a pH-adjuster (i.e. acid or base) or its salts, a co-solvent and a surfactant.

1. Water

The amount of water present in the solvent system or composition according to the present invention is of utmost importance in aspect of the applicability and in aspect of its administration to patients. In compositions where the therapeutically effective amount of pharmaceutically active agent cannot be dissolved in water alone, adequate amounts of solubilizing excipients such as co-solvents or surfactants can be used. It is well known for a skilled person in the art that the amount of organic or non-aqueous solvents or excipients replacing water in a dosage form [especially in parenteral (intravenous) dosage forms] is limited due to the toxicity of these excipients. The determination of the amount of the water to be present in a composition is based on benefit/risk assessments (taken into consideration the composition of previously registered products). Of course, a skilled person intends to apply as little amount of toxic excipients as possible. Based on such considerations it is proposed that the solvent systems according to the present invention should comprise 1-99% water, preferably 50-90% water, more preferably 60-80% by volume water. In case of parenteral administration, of course, the water is preferably a so-called “water for injection”.

2. pH-Adjuster

The term pH-adjuster relates to an acid or base, or their salts or a mixture thereof throughout the entire specification. An acid or a base according to Bronsted-Lowry is a molecule capable of donating or accepting a proton (hydrogen ion), respectively. Due to their previously stated property, acids and bases adjust the pH of the solution in which they are in to 0-7, if they are acidic, and to 7-14, if they are basic. Without getting bound to a theory it can be said that the solubility of weakly acidic or basic compounds depends mainly on their intrinsic solubility, pKa and the pH of its environment. This can be expressed by the Henderson-Hasselbach equation. Therefore, the solubility of drugs with ionizable groups can be considerably altered by means of pH adjustment. It is known to a skilled person that the acceptable range for parenteral administration is determined by the buffer capacity of the blood and preferably it is pH 3-8. Any injectable composition having a pH outside this range may cause irritation, tissue damage and pain on administration; therefore it should be administered with care. Preferably, a pharmaceutical composition falling into the preferred range should be formulated. Based on the previous theory the acids and bases used in the present invention include, but are not limited to organic or inorganic acids or bases, acids or bases which are salts of pharmaceutically acceptable bases or acids, respectively, and the mixtures thereof. More preferably, the acids include, but are not limited to boric acid, hydrochloric acid, hydrobromic acid, hydroiodic acid, nitric acid, phosphoric acid, sulfuric acid, acetic acid, amino acids, ascorbic acid, benzoic acid, carbonic acid, citric acid, formic acid, gluconic acid, lactic acid, maleic acid, propionic acid, salicylic acid, tartaric acid, thioglycolic acid and uric acid. In the present invention salts of the preferred acids also can be applied, where the cationic part of them can be, but not limited to ammonium, calcium, lithium, magnesium, potassium and sodium ion. Bases used in the present invention are, but not limited to hydroxides, carbonates, hydrocarbonates, silicates of the previously listed cations and triethanolamin and their analogues. It should be understood that the list is merely illustrative and any analogue or derivative or a mixture of the stated molecules are within the scope of the present invention.

3. Co-Solvents

Co-solvents are pharmaceutically acceptable excipients used beside water to solubilize poorly soluble molecules. Co-solvents are added to the aqueous solution at a chosen percentage and thus the dielectric constant of the resulting solvent systems decreases. This phenomenon and the ability of the co-solvents to disrupt the secondary bonding structure of water results in their capability of solubilizing poorly soluble therapeutically active agents. These solvents are mainly organic and as such have to be used with certain limitations due to their ability to cause precipitation, inflammation and pain at the site of injection. As already discussed above, to avoid such side effects the lowest possible, but suitable amount of excipient is required. In an embodiment of the present invention the co-solvents are for example alcohols, preferably C1-C6 alcohols, more preferably C1-C4 alcohols, even more preferably ethyl-alcohol, propylene glycol, glycerol; ethers, more preferably ethers of polyethylene glycols, even more preferably ethers of polyethylene glycols of molecular mass 200-6000, even more preferably polyethylene glycol 400; amides; esters and other solubilizers known in the art. It should be understood that the list is merely illustrative and any analogue or derivative or a mixture of the mentioned molecules are within the scope of the present invention.

The amount of the co-solvent is 1-80%, preferably 10-50%, more preferably 15-30% by volume.

4. Surfactants

Surfactants are a valuable group of excipients also used in water for the solubilization of therapeutically active hydrophobic entities, more preferably sparingly, slightly, very slightly soluble or practically insoluble, or non-soluble therapeutically active agents. To serve as a surfactant the compound must have a hydrophilic and a hydrophobic part. Based on their structure one can differentiate non-ionic, ionic and hydrophobic surfactants, but irrelevant of their structure all surfactants can form micelles in an aqueous solution if they are present in an adequate amount. These micelles are then capable of solubilizing molecules either by entrapping the molecule in the micelle or by other means of bonding to the therapeutically active agent. In a embodiment of the present invention the surfactant can be from the group of polyethoxylated fatty acids, PEG fatty acid esters, polyethylene glycol glycerol fatty acid esters, alcohol-oil transesterification products, polyglycerized fatty acids, propylene glycol fatty acid esters, mono- and diglycerides, sterol and sterol derivatives, polyethylene glycol sorbitan fatty acid esters, polyethylene glycol alkyl ethers, sugar esters and sorbitan fatty esters or ionic surfactant. More preferably from the group of polyethylene glycol sorbitan fatty acid esters or ionic surfactant, even more preferably polysorbates or a derivative of choleate, even more preferably polysorbate 20, 60, 80, most preferably polysorbate 80, or sodium taurocholeate or a combination thereof. It should be understood that the list is merely illustrative and any analogue or derivative or a mixture of the mentioned molecules are within the scope of the present invention.

The amount of the surfactant can be 0.01 to 10%, preferably 0.1 to 5%, most preferably 3% by weight.

### EXAMPLES

The following examples illustrate and describe the present invention through some preferred embodiments without the intention to limit the scope claimed.

General example for the solubility tests

Each of the solutions applied in the solubilization tests were prepared as follows (the specific amount of the components comes from the Tables below):

1. 0.05M ammonium acetate (as pH-adjuster) solution was prepared in a volumetric flask and the pH of this solution was set to pH 3.1 with glacial acetic acid.

2. The specific amount of co-solvent was measured into a beaker and the specific amount of the previously prepared 0.05M ammonium acetate solution was added. The pH of this solution was measured and set to pH 3.1 with glacial acetic acid.

3. The specific amount of surfactant was added to the solution.

4. This solvent system was vigorously shaken until complete homogenization of the solution was achieved.

5. Aliquot amount of this solvent system was measured and the specific amount of the active agent was added to the solution.

The Tables below define the composition of each solution tested and the amount of the active agent which could be solubilized by the applied solvent system.

The amount of the co-solvent is given in % by volume and the amount of the surfactant is given in % by weight in each table.

Synergistic Effect

In order to justify our previous statement, according to which the solvent system exhibits a synergistic solubilizing effect, the solubility of miconazole was measured separately in pH adjusters, co-solvents and surfactants, i.e. in the components of the solvent system. The results are given in Table 1. These data can be used for comparison with Tables 2 and 3 wherein the results were obtained by the solvent system according to the invention.

The synergistic effect means that the solubility enhancer effect of the combination applied [pH-adjuster(s), co-solvent(s) and surfactant(s)] is more than the sum of the solubilizing effect of each excipient applied separately. Tables 2 and 3 show the solubilizing effect of various combinations of a pH adjuster, co-solvent(s) and surfactant(s). As it can be seen, surprisingly, adding the solubilized amount of miconazole in 0.05 M ammonium acetate, in 25% ethanol and in 3% polysorbate gives a much lower value than the measured value in case of the solvent system where the mentioned components are applied together.

TABLE 1
The effect of pH-adjusters, co-solvents and surfactants
separately on the solubility of miconazole

| Solubilizer in water at | Solubilized miconazole ± |
|---|---|
| given concentration | S.D. (mg/ml) |
| 0.05 M ammonium acetate pH 3.1 | 2.24 ± 0.36 |
| 0.05 M calcium gluconate pH 3.1 | 0.65 ± 0.06 |
| 0.05 M potassium dihydrogen | 0.77 ± 0.05 |
| phosphate pH 3.1 | |
| 30% glycerol | 0.024 ± 0.006 |
| 30% macrogol 400 | 0.022 ± 0.008 |
| 60% propylene glycol | 0.36 ± 0.12 |
| 25% ethanol | 0.021 ± 0.01 |
| 3% polysorbate 20 | 0.81 ± 0.07 |
| 3% polysorbate 60 | 1.42 ± 0.10 |
| 3% polysorbate 80 | 1.52 ± 0.09 |

Combination of pH Adjuster(s), Co-Solvent(s) and Surfactant(s)

As it was discussed in the detailed description of the invention, it is advantageous to reduce the amount of excipients in a parenteral dosage form as much as possible. Tables 2 and 3 show the results of the experiments performed to comply with these requirements. Table 2 shows the results of the study of the effect of various concentrations of polysorbate 80 on the solubility of miconazole in a solvents system comprising polysorbate 80, 25% ethanol and ammonium acetate at pH 3.1.

TABLE 2
Concentration of solubilized miconazole in compositions containing
25% ethanol as co-solvent, ammonium acetate as pH-adjuster (pH
3.1) and varying amounts of polysorbate 80 as surfactant.

| | Solubilized miconazole ± |
|---|---|
| Composition | S.D. (mg/ml) |
| 5.0% polysorbate 80 + 25% ethanol + | 42.68 ± 0.81 |
| ammonium acetate pH 3.1 | |
| 4.0% polysorbate 80 + 25% ethanol + | 43.26 ± 2.61 |
| ammonium acetate pH 3.1 | |
| 3.0% polysorbate 80 + 25% ethanol + | 38.92 ± 0.61 |
| ammonium acetate pH 3.1 | |
| 2.0% polysorbate 80 + 25% ethanol + | 37.93 ± 1.86 |
| ammonium acetate pH 3.1 | |
| 1.0% polysorbate 80 + 25% ethanol + | 42.58 ± 2.04 |
| ammonium acetate pH 3.1 | |
| 0.50% polysorbate 80 + 25% ethanol + | 44.72 ± 0.14 |
| ammonium acetate pH 3.1 | |
| 0.25% polysorbate 80 + 25% ethanol + | 34.39 ± 1.51 |
| ammonium acetate pH 3.1 | |
| 0.10% polysorbate 80 + 25% ethanol + | 35.99 ± 2.09 |
| ammonium acetate pH 3.1 | |

As it can be seen, surprisingly, the obtained solubility results showed that reducing the quantity of the surfactant (polysorbate 80) did not result in substantial loss of solubilizing capability. In the next phase of the experiments the amount of the co-solvent (ethanol) was reduced. The solubility test results of miconazole are shown in Table 3 obtained in a solvent system comprising 0.1% polysorbate 80, various concentrations of ethanol and ammonium acetate (pH 3.1).

TABLE 3
Concentration of solubilized miconazole in compositions
containing 0.1% polysorbate 80, ammonium acetate adjusted
to pH 3.1 and varying amounts of ethanol.

| | Solubilized miconazole ± |
|---|---|
| Composition | S.D. (mg/ml) |
| 0.1% polysorbate 80 + 25% ethanol + | 35.99 ± 1.49 |
| ammonium acetate pH 3.1 | |
| 0.1% polysorbate 80 + 20% ethanol + | 35.05 ± 5.76 |
| ammonium acetate pH 3.1 | |
| 0.1% polysorbate 80 + 15% ethanol + | 10.79 ± 2.84 |
| ammonium acetate pH 3.1 | |
| 0.1% polysorbate 80 + 10% ethanol + | 4.52 ± 0.03 |
| ammonium acetate pH 3.1 | |
| 0.10% polysorbate 80 + 5% ethanol + | 2.39 ± 0.12 |
| ammonium acetate pH 3.1 | |

As it can be seen, reducing the amount of the co-solvent ethanol from 25% to 20% had negligible effect but further decreasing had significantly negative effect on the solubilizing capability of the solvents system. It should also be noted that further increasing the amount of ethanol is not advisable on toxicological issues previously discussed.

These compositions were autoclaved and showed excellent stability. Here we would mention that the sterility can be achieved by membrane filtration (applying proper pore size and compatible conditions).

Stability on Dilution with Infusions

A composition called as “concentrate for infusion” should be diluted before administration to patients by a previously determined amount of usual infusion. The usual infusion is chosen preferably from the group of 5% dextrose infusion and 0.9% sodium chloride infusion. It is well known for a skilled person in the art that such a dilute should be stable for at least 48 hours. To reproduce the dilution scheme applied in the case of Monistat® (previously marketed as a concentrate for infusion containing miconazole), one of the compositions of the present invention was diluted with 5% dextrose infusion and 0.9% sodium chloride infusion. Based on the previous results a composition comprising of 0.1% polysorbate 80, 20% ethanol and ammonium acetate pH 3.1 was chosen for further investigation. 10 ml of the chosen composition containing 20 mg/ml of miconazole was diluted to 200 ml with 5% dextrose infusion or 0.9% sodium chloride infusion. Since this composition was not stable for 48 hours, further investigations were performed. It was found that, depending on the amount of miconazole solubilized in the composition, various concentrations of surfactants are necessary to keep the active agent solubilized on dilution. Table 4 shows the amount of surfactant necessary to solubilize various concentrations of miconazole on dilution to 200 ml with 5% dextrose or 0.9% sodium chloride infusions.

TABLE 4
Concentration of polysorbate 80 necessary to keep in solution
various concentrations of miconazole on dilution to 200 ml
with 5% dextrose or 0.9% sodium chloride infusions.

| Concentration of | Concentration of polysorbate 80 (co-solvent |
|---|---|
| miconazole in composition | 20% ethanol + ammonium acetate, pH 3.1) |
| 10 mg/ml | 0.1% |
| 20 mg/ml | 3% |
| 30 mg/ml | 3% |

On the basis of the above results it was established that concentration of the surfactant can be 0.01 to 10%, preferably 0.1 to 5%, most preferably 3%.

Further Compositions Showing Synergistic Effect

Table 5 contains further solubilizing compositions and the measured concentrations of miconazole, ketoconazole and itraconazole. The compositions presented below comprise of 3% surfactant, 25% co-solvent and pH adjuster (pH 3.1). The first composition is thought to be the most suitable for miconazole. Further compositions were prepared to find solvent systems showing even better solubilizing capability.

TABLE 5
Compositions showing synergistic effect on the solubility
of miconazole and ketoconazole, but not on itraconazole.

| | Solubilized | Solubilized | Solubilized |
|---|---|---|---|
| | miconazole ± | ketoconazole ± | itraconazole ± |
| Composition | S.D. (mg/ml) | S.D. (mg/ml) | S.D. (mg/ml) |
| 3% polysorbate 80 + 25% | 34.12 ± 2.95 | 139.43 ± 4.05 | 0.057 ± 0.005 |
| ethanol + ammonium acetate | | | |
| pH 3.1 | | | |
| 3% polysorbate 80 + 15% | 21.99 ± 1.62 | 140.50 ± 3.99 | 0.054 ± 0.002 |
| ethanol + 10% PEG 400 + | | | |
| ammonium acetate pH 3.1 | | | |
| 3% polysorbate 80 + 15% | 10.67 ± 0.69 | 124.65 ± 1.16 | 0.040 ± 0.001 |
| ethanol + 10% propylene | | | |
| glycol + ammonium acetate | | | |
| pH 3.1 | | | |
| 1.5% sodium taurocholeate + | 32.10 ± 2.86 | 137.15 ± 8.15 | 0.040 ± 0.008 |
| 1.5% polysorbate 80 + 25% | | | |
| ethanol + ammonium acetate | | | |
| pH 3.1 | | | |
| 1.5% sodium taurocholeate + | 34.80 ± 2.36 | 141.58 ± 2.85 | 0.040 ± 0.001 |
| 1.5% polysorbate 80 + 15% | | | |
| ethanol + 10% PEG 400 + | | | |
| ammonium acetate pH 3.1 | | | |
| 1.5% sodium taurocholeate + | 38.38 ± 1.53 | 126.54 ± 1.04 | 0.026 ± 0.001 |
| 1.5% polysorbate 80 + 15% | | | |
| ethanol + 10% propylene | | | |
| glycol + ammonium acetate | | | |
| pH 3.1 | | | |

As it can be seen, very surprisingly, although all three compounds are azole derivatives and show significant similarity in respect of their chemical structure but their solubility differs greatly in the compositions listed above. Ketoconazole showed the best solubility in the compositions, followed by miconazole, which also showed a remarkable solubility. The amounts of both of these active agents solubilized in the compositions are therapeutically significant and can be conveniently applied to formulate a concentrate for infusion. On the other hand, itraconazole showed no substantial solubility in the compositions of the present invention.

Comparison with U.S. Pat. No. 6,383,471.

U.S. Pat. No. 6,383,471 discloses several compositions comprising a hydrophobic therapeutically active agent containing at least one ionizable functional group, an ionizing agent, a solubilizer and a surfactant and the method of producing the same. A few examples using itraconazole and tretinoin as the hydrophobic therapeutically active agent are disclosed and their stability upon dilution with gastric and intestinal fluid is also thought. Examples 27-31 are detailed in the background of the invention. It is also disclosed in the patent that compositions 27 to 31 are capable of dissolving 85, 50, 50, 30 and 30 mg/ml of itraconazole respectively. These compositions comprise no water and, as stated before, the amount of water present in a pharmaceutical composition, especially in parenteral dosage forms, is of utmost importance. No data concerning the stability of these compositions on autoclaving or membrane filtration are given and parenteral route of administration is one of the least preferable ways of administration according to the patent. Present invention describes solvent systems exerting synergistic solubilizing effect on hydrophobic azole derivatives. In order to compare the solubilizing effect of the solvent system of the present invention and the same of U.S. Pat. No. 6,383,471, the solvent systems of Examples 27-31 of this patent were reproduced. Table 6 shows the data of obtained by these solvent systems in case of miconazole, ketoconazole and itraconazole.

TABLE 6
Solubilized miconazole, ketoconazole and itraconazole
in solvent systems of Examples 27-31

| Solving system | Solubilized | Solubilized | Solubilized |
|---|---|---|---|
| (No. of | miconazole ± S.D. | ketoconazole ± S.D. | itraconazole |
| example) | (mg/ml) | (mg/ml) | (mg/ml)* |
| 27 | 64.66 ± 5.43 | 49.38 ± 1.47 | 85 |
| 28 | 39.05 ± 5.53 | 60.96 ± 3.28 | 50 |
| 29 | 51.17 ± 2.07 | 52.26 ± 3.25 | 50 |
| 30 | 91.21 ± 5.74 | 105.02 ± 2.76 | 30 |
| 31 | 84.06 ± 5.63 | 113.43 ± 3.12 | 30 |

*data from U.S. Pat. No. 6,383,471

The solubilizing effect of the solvent system of the present invention is shown in Table 5. It should be noted again that the compositions according to the present invention contain preferably about 70% of water. To have a proper base for comparison with the compositions of U.S. Pat. No. 6,383,471 they should be diluted with water to 70% of water content. The accordingly calculated solubilizing effect of solving systems of examples 27-31 from U.S. Pat. No. 6,383,471 are shown in Table 7.

TABLE 7
Calculated solubilizing effect of compositions
27-31 diluted to 70% of water content

| Solving system | Solubilized | Solubilized | Solubilized |
|---|---|---|---|
| (No. of | miconazole | ketoconazole | itraconazole |
| example) | (mg/ml) | (mg/ml) | (mg/ml)* |
| 27 + 70% water | 19.49 | 14.81 | 25.5 |
| 28 + 70% water | 11.71 | 18.28 | 15 |
| 29 + 70% water | 15.35 | 15.68 | 15 |
| 30 + 70% water | 27.36 | 31.56 | 9 |
| 31 + 70% water | 25.21 | 34.03 | 9 |

*calculated on the basis of data of U.S. Pat. No. 6,383,471

Comparing the data of Table 7 with the data of Table 5 it can be seen that the solvent systems of the present invention exert a greater solubility increase in the case of miconazole and a surprisingly much greater solubility increase in the case of ketoconazole. It is also shown that although itraconazole shows substantial chemical similarity to miconazole and ketoconazole, surprisingly, the compositions of the present invention do not exert a substantial solubility enhancing effect on its solubility, compared to the compositions in U.S. Pat. No. 6,383,471.

Summarizing the results of the invention it can be concluded that the compositions of the present invention exert a substantial synergistic solubilizing effect on a group of pharmaceutically active agents, namely on antifungicide azole derivatives. The compositions of the present invention comply with all the requirements set against parenteral (preferably injectable) compositions. In more detail, the solvent system contains water which, as it was detailed above, is a very important component in parenteral dosage forms. All the other components mentioned above are pharmaceutically acceptable in parenteral dosage forms. This acceptability also concerns the quantity of applied components. It is also shown that the compositions of the present invention can be sterilized either by autoclaving or sterile membrane filtration. It is also shown that the compositions can be diluted into large volume and these diluted compositions are applicable in parenteral administration.

1. A pharmaceutical composition comprising:
an azole derivative solubilized in a solvent system comprising:
a) water
b) pH-adjuster,
c) co-solvent and
d) surfactant.

2. A pharmaceutical composition according to claim 1, which comprises 1-99%, preferably 50-90%, more preferably 60-80% by volume of water.

3. A pharmaceutical composition according to claim 1, which comprises a pH-adjuster buffering in the range of pH 3-8.

4. A pharmaceutical composition according to claim 1, which comprises a C1-C6 alcohol, more preferably C1-C4 alcohol as co-solvent.

5. A pharmaceutical composition according to claim 1, which comprises a polyethylene glycol sorbitan fatty acid ester as surfactant.

6. A pharmaceutical composition according to claim 1, wherein the pH-adjuster is ammonium acetate in a solution of pH 3.1 adjusted by acetic acid, the co-solvent is ethanol and the surfactant is a polysorbate, preferably polysorbate 80.

7. A pharmaceutical composition according to claim 1, wherein the azole derivative is miconazole or ketoconazole.

8. A solvent system for solubilizing azole derivatives comprising:
a) water
b) pH-adjuster,
c) co-solvent and
d) surfactant.

9. A solvent system according to claim 8, which comprises 1-99%, preferably 50-90%, more preferably 60-80% by volume of water.

10. A solvent system according to claim 9, which comprises a pH-adjuster buffering in the range of pH 3-8.

11. A solvent system according to claim 8, which comprises a C1-C6 alcohol, more preferably C1-C4 alcohol as co-solvent.

12. A solvent system according to claim 8, which comprises a polyethylene glycol sorbitan fatty acid ester as surfactant.

13. A solvent system according to claim 8, wherein the pH-adjuster is ammonium acetate in a solution of pH 3.1 adjusted by acetic acid, the co-solvent is ethanol and the surfactant is a polysorbate, preferably polysorbate 80.

14. Method of treatment of animals, more preferably mammals, even more preferably humans, characterized by administering to the subject to be treated a therapeutically effective amount of a composition according to claim 1.
